(19) United States
(12) Patent Application Publication
(10) Pub. No.: US 2022/0288288 A1
(43) Pub. Date: Sep. 15, 2022

(54) BAG SYSTEM FOR COLLECTING BLOOD FROM ANIMALS

(71) Applicant: Maco Pharma, Mouvaux (FR)

(72) Inventors: Martin ROBIQUET, Mouvaux (FR); Raphael Gavroy, Mouvaux (FR)

(21) Appl. No.: 17/633,596

(22) PCT Filed: Aug. 10, 2020
(86) PCT No.: PCT/EP2020/072342

§ 371 Date: Feb. 8, 2022

(30) Foreign Application Priority Data
Aug. 9, 2019 (FR) FR1909101

(51) Int. Cl. A61M 1/02; A61M 1/00

(52) U.S. Cl. CPC A61M 1/0236; A61M 1/70; A61M 2250/00

(57) ABSTRACT

Bag system (1) for collecting blood from an animal, comprising a collection bag (2) fluidically connected, via a first tubing (4) associated with an inlet tubing of the collection bag (2), to a first sampling needle (3), a second sampling needle (4) being connected via a second tubing (6), said second sampling needle (5) having an outer diameter that is different from the first sampling needle (3).

The invention relates to a bag system for collecting blood from animals.

The invention lies in the field of blood transfusions in animals, more particularly for dogs and/or cats.

As is the case for humans, animals can benefit from blood transfusions in order to treat illnesses such as anaemia, where there is a significant reduction in the haemoglobin level or the number of red blood cells in the blood, or to treat problems relating to the coagulation process.

Blood transfusions define the administration of a blood product from a healthy donor animal to a recipient animal. Most commonly, a fresh blood transfusion is performed, with blood being collected from the donor and then administered to the recipient within 24 hours.

In the case of dogs, they can be qualified as a donor if they meet certain criteria.

In order to donate blood, the dog must be in good health, between 2 and 8 years old, have up-to-date vaccinations and generally be large, i.e. weighing more than 35 kilograms. However, some veterinarians collect blood from smaller dogs, from 20 kilograms.

Currently, there are different ways to collect blood from the canine species.

The first solution is to collect whole blood directly into syringes. This is called an “open” collection system.

This method requires the blood to be transfused immediately. The blood should never be stored as there is a high risk of contamination and the life span of red blood cells and platelets is reduced.

Other open systems exist, where a bag or syringe, a three-way valve and a needle must be connected together, and an anticoagulant and a preservative must be injected into the bag. All these operations must be carried out under the most sterile conditions possible.

This type of system inevitably exposes the collection procedure to the risk of bacterial contamination during the various steps.

Lastly, and in the majority of cases, the practitioner uses a “closed” bag system, consisting of a sampling needle connected to a collection bag by means of a tubing. This method allows the blood to be collected in a sterile manner and stored away from air and contaminants.

The disadvantage of these bag systems, which are generally used for human blood collection, is that they are mainly suitable for large dogs, which limits collection procedures in smaller dogs.

In addition, when such a system is used to take blood from a small dog, the needle, which is a standard size, is too large. This results in discomfort for the animal during blood donation, as well as a longer healing time compared to larger dogs.

The invention aims to overcome this problem by providing a bag system that allows the blood collection procedure to be performed more comfortably on an animal of any size.

In addition, bag systems comprising two identically sized sampling needles are known, for example from EP 1 262 202. These systems are specifically intended for the collection of cord blood and allow the umbilical cord to be pricked at two different locations in order to increase the volume of blood collected.

Also known from US 2016/0279320 is a medical device for the preparation of platelet-rich plasma from whole blood. This medical device comprises a set of three modules to be connected together comprising bags, syringes, tubes, connectors and needles. One of the modules comprises a blood sampling needle and a needle for removing an anticoagulant from a container. In order to adapt this medical device to the patient to be treated—child or adult—it is foreseen to provide different appropriate modules.

The invention relates to a bag system for collecting blood from an animal, comprising a collection bag fluidically connected to a first sampling needle via a first tubing associated with an inlet opening of the collection bag, the bag system comprising a second sampling needle connected to said first tubing via a second tubing, the second sampling needle having an outer diameter different from that of the first sampling needle.

Further objects and advantages will become apparent in the course of the following description, which is provided with reference to the attached drawings, in which:

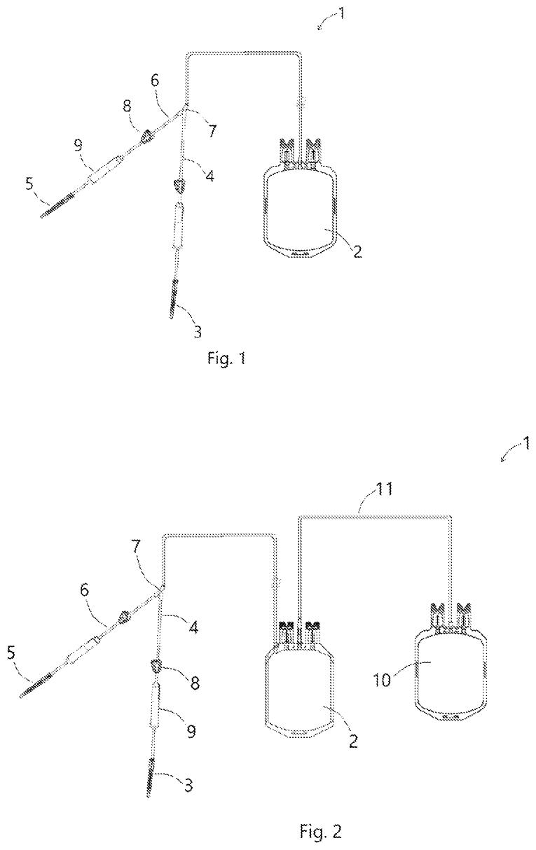

FIG. 1 shows a schematic front view of a first embodiment of a bag system according to the invention.

FIG. 2 shows a schematic front view of a second embodiment of a bag system according to the invention.

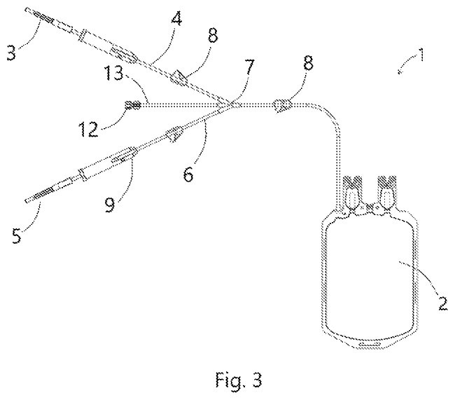

FIG. 3 shows a schematic front view of a third embodiment of a bag system according to the invention.

The invention relates to a bag system typically intended to perform blood collection in an animal and more particularly in a dog.

In relation to FIGS. 1 to 3, the bag system (1) comprises a collection bag (2) fluidically connected to a first sampling needle (3) via a first tubing (4) associated with an inlet opening of the collection bag (2).

The collection bag (2) is also fluidically connected to a second sampling needle (5) via a second tubing (6) generally connected to the first tubing (4).

The first and second sampling needles (3, 5) are adapted to prick an animal in its jugular vein to cause blood to flow by gravity directly into the collection bag.

According to the invention, the second sampling needle (5) has a different external diameter than that of the first sampling needle (3).

The diameter of the sampling needles (3, 5) is expressed in gauge, which is the unit of measurement for needles. The gauge defines how many needles of the same type can be contained in one square inch. Thus, the larger the gauge, the smaller the outer diameter of the needle.

According to one embodiment, each of the needles is a needle ranging from 13 to 20 gauges, i.e. having an outer diameter ranging from 2.5 mm to 0.7 mm. Each of the needles is a needle ranging from 15 to 17 gauges, i.e. having an outer diameter ranging from 2.5 mm to 1.5 mm.

In particular, one of the sampling needles (3, 5) is a gauge 16 needle or less, i.e. with an outer diameter of 1.6 mm or more. For example, it has a length of 50 mm and/or an inner diameter of 1.5 mm or more.

More particularly, one of the two sampling needles (3, 5) is a 17 gauge needle or larger, i.e. with an outer diameter of 1.5 mm or less. For example, it has a length of 45 mm and/or an inner diameter of 1.3 mm or less.

Even more particularly, the bag system (1) comprises a first sampling needle (3) of gauge 16 and a second sampling needle (5) of gauge 17.

In one embodiment, the second tubing (6) associated with the second sampling needle (5) is connected to the first tubing (4) via a junction (7), for example a T or Y junction.

The presence of two needles (3, 5) of different sizes is advantageous in that it allows the veterinarian to choose the right needle for the size and comfort of the animal.

Each of the sampling needles (3, 5) consists of a cannula and a hub.

The cannula has a hollow tube and ends in a tip. The opposite end of the tip of the cannula forms the base of the cannula. The tip of the cannula is in particular bevelled. Such a cannula is particularly intended for piercing the skin of the animal.

In particular, the cannula is made of metal and coated with silicone to ensure better insertion.

The cannula is held at its base in a hub, which facilitates the gripping of the sampling needle (3, 5). The hub is for example made of a rigid, sterilisable thermoplastic material such as polycarbonate.

In order to avoid both the risk of accidental pricking during handling and also the risk of damage to the tip of the cannula before use, it is customary to cover the cannula with a protective device, such as a cap. This protective device also ensures the sterility of the cannula.

The collection bag (2) is intended to contain the blood collected from the donor animal.

The collection bag is formed by two plastic sheets joined, for example by welding, on their outer periphery so as to define an inner volume. The collection bag (2) further comprises one or more inlet and/or outlet openings.

The sheets are made of flexible, biocompatible, weldable and sterilisable plastic material, for example polyvinyl chloride.

The inlet and/or outlet openings, formed in particular by tubing portions, are set up to receive tubing in a sealed manner or are sealed in a sterile manner by a device which can be torn off in the event that the operator wishes to use them.

Advantageously, the collection bag (2) is pre-filled with an anticoagulant and/or preservative solution.

In particular, the anticoagulant and/or preservative solution is of the CPDA1 (Citrate Phosphate Dextrose Adenine) type.

The tubing is also made of flexible, biocompatible, weldable and sterilisable material, for example polyvinyl chloride.

The bag system also comprises one or more means for selectively opening/closing the fluid connection through the tubing (4, 6), for example in the form of a clamp (8).

In fact, depending on the sampling needle (3, 5) used for blood collection, the practitioner will have to prevent fluid flow in the tubing (4,6) connected to the unused sampling needle (3, 5) by closing the clamp (8).

The bag system (1) further comprises needle guards (9) formed from a hollow cylinder and slidably mounted on the tubing (4, 6) connecting the sampling needles (3, 5) to the collection bag (2).

After use, the sampling needle (3, 5) is covered by the needle guard (9) in order to prevent a risk of pricking.

In relation to FIG. 2, the bag system (1) further comprises a transfer bag (10), fluidically connected to the collection bag (2) via a third tube (11), so as to form a closed system.

In this embodiment, at the end of the collection process and after centrifugation of the bag system and separation, separate blood components such as plasma and a concentrate of red blood cells are obtained in each bag (10, 2).

The bag system (1) is advantageously sterilised to avoid contamination of the collected blood.

In an embodiment shown in FIGS. 1 and 2, the bag system is closed, i.e. the sampling needles (3, 5), tubing (4, 6, 11) and bags (2, 10) are pre-connected from the factory. The blood collection and processing are thus carried out in a closed circuit and in a sterile manner in order to avoid any bacterial contamination of the collected blood.

With reference to FIG. 3, a further embodiment of the bag system (1) of the invention is described.

In this embodiment, the bag system (1) further comprises a device (12) for connection to a third sampling needle (not shown). This connection device (12), which may be a Luer fitting, is fluidically connected to the collection bag (2) via a fourth tubing (13) generally connected to the first tubing (4) via a junction (7).

In particular and according to this embodiment, the junction (7) is a 4-way connector.

The third sampling needle has a different outer diameter than that of the first and/or second sampling needle (3, 5).

Thus, if the practitioner wishes to collect blood from an animal using a sampling needle of a different size than the two sampling needles (3, 5) already present in the bag system (1), he connects the needle of his choice to the connection device (12).

A method for collecting blood from an animal using a bag system (1) according to the invention is described below.

In a first step, the collection site, usually at the jugular vein in the case of a dog because the blood flow is greater there, is disinfected to avoid contamination of the blood and prevent the risk of phlebitis for the donor animal.

The veterinarian then selects the sampling needle (3, 5) appropriate for the size of the animal and pierces the vein.

In particular, when collecting blood from a dog, if it is a large dog, the larger sampling needle (3), usually 16 gauge, is preferably used. If the dog is a small dog, the smaller sampling needle (5), usually 17 gauge, is used.

The tubing (4, 6) connecting the non-used sampling needle to the collection bag (2) is closed by means of the corresponding clamp (8).

When the clamp (8) on the tubing connecting the used needle to the collection bag is opened, the blood flows by gravity into the collection bag (2).

The blood is collected in the collection bag (2) and homogenised with the anticoagulant placed beforehand in the collection bag (2), either by a user gently stirring the collection bag (2) or by an agitator, in order to avoid clot formation.

The collection procedure generally yields 450 ml of blood in large dogs and 200-300 ml of blood in small dogs.

Once the required volume of blood has been collected, the practitioner replaces the clamp (8) on the tubing (4, 6) next to the sampling needle (3, 5) and closes it to prevent air from entering the collection bag (2). The sampling needle (3, 5) is then withdrawn from the vein and a compress is placed over the collection site of the donor animal.

The collection bag (2) is then sealed by cutting the tubing (4, 6) as close to the collection bag (2) as possible using a hand-held sealer.

The collection bag (2) is identified with the date of collection, the name of the donor, the donor group, the amount of blood contained in the collection bag (2) and the type of anticoagulant used.

Lastly, the collected blood is either transfused immediately, stored in a refrigerator for three weeks, or centrifuged to separate the red blood cells from the plasma immediately after collection.

In the case of centrifugation, the plasma is transferred to a transfer bag (10) directly fluidically connected to the collection bag (2).

1. A bag system (1) for collecting blood from an animal, comprising a collection bag (2) fluidically connected on the one hand to a first sampling needle (3) via a first tubing (4) associated with an inlet tubing of said collection bag (2), and on the other hand to a second sampling needle (5) via a second tubing (6), characterised in that said second sampling needle (5) has an outer diameter that is different from that of the first sampling needle (3).

2. The bag system (1) according to claim 1, characterised in that one of the sampling needles (3, 5) is a 16 gauge needle or less.

3. The bag system (1) according to claim 1, characterised in that one of the sampling needles (3, 5) is a 17 gauge needle or more.

4. The bag system (1) according to claim 1, characterised in that the first sampling needle (3) is a 16 gauge needle and the second sampling needle (5) is a 17 gauge needle.

5. The bag system (1) according to claim 1, characterised in that the second tubing (6) is connected to the first tubing (4) via a junction (7).

6. The bag system (1) according to claim 1, characterised in that each of the first and second tubings (4,6) comprises a means for selectively opening and closing (8) the fluidic connection through said tubing (4,6).

7. The bag system (1) according to claim 1, characterised in that the collection bag (2) comprises an anticoagulant and/or preservative solution.

8. The bag system (1) according to claim 1, characterised in that it also comprises a device (12) for connection to a third sampling needle, in fluidic connection to the collection bag (2) via a fourth tubing (13).

9. The bag system (1) according to claim 1, characterised in that the bag system (1) also comprises a transfer bag (10), fluidically connected to an outlet opening of the collection bag (2) via a third tubing (11).
